(19) United States
(12) Patent Application Publication
(10) Pub. No.: US 2021/0267625 A1
(43) Pub. Date: Sep. 2, 2021

(54) Skin Growth Excision Apparatus

(71) Applicant: Tag Off LLC, Wheat Ridge, CO (US)

(72) Inventor: Alan R. Carver, Erie, CO (US)

(73) Assignee: Tag Off LLC, Wheat Ridge, CO (US)

(21) Appl. No.: 16/804,931

(22) Filed: Feb. 28, 2020

(51) Int. Cl. A61B 17/3209

(52) U.S. Cl. CPC A61B 17/32093; A61B 2017/00761

(57) ABSTRACT

A skin growth excision apparatus, and methods of making and using such a skin growth excision apparatus, whereby the skin growth excision apparatus can include a base having opposing base upper and lower surfaces, an aperture element communicating between the base upper and lower surfaces, and a blade overlaying the base upper surface. The blade can be slidably engaged with the base to facilitate travel of the blade along a travel path over the aperture element, whereby the blade can excise a skin growth extending therethrough.

### I. BACKGROUND

The instant invention enables self-treatment of a skin growth extending from the skin surface. Although not limited to such, this invention may be useful for the excision of an acrochordon (commonly called a skin tag), which can be a relatively small growth often found in areas where the skin creases, such as the eye lid, neck, armpit, or groin area. Typically, skin tags are harmless and painless, and tend not to grow or change over time. While skin tags of over half an inch in length have been reported, they tend to be about the size of a grain of rice and may have either a smooth or an irregular surface. It is believed that slightly less than half of the population has at least one skin tag.

Generally, skin tags are benign, and many people simply ignore them. However, some desire to have their skin tags removed for cosmetic reasons. For removal, one can visit a dermatologist or other medical professional for excision, cauterization, cryosurgery, or surgical ligation of the skin tag. Normally, skin tag removal is performed in a doctor's office, often with little more than a topical anesthetic placed at the skin tag site and a bandage placed on the wound after removal.

Removal of a skin tag by a medical professional is a relatively minor procedure; nevertheless, some people do not want to go through the burden or expense of a doctor's visit and opt to remove the skin tag themselves, for example using a readily available cutting implement such as a razor blade, scissors, or nail clippers. While such a “home remedy” may avoid a visit to the doctor's office, it may not be without its drawbacks. For example, if the cutting implement is not sterile, a person runs the risk of contracting an infection at the wound site. Additionally, a person may miss the skin tag with the cutting implement, either partially or completely, or may cut deeper than necessary into the skin, thereby creating a larger-than-necessary wound which may require stitching for closure. Such misses or deep cuts can be especially problematic when dealing with a skin tag located on an eye lid or otherwise near an eye or other vital body part.

Accordingly, a method and corresponding apparatus are needed which allow for relatively quick and easy removal of a skin tag from the body while avoiding the above-detailed shortcomings which may be associated with in-home skin tag removal. Specifically, such an apparatus should be sterile so as avert contraction of an infection by a user. Further, such an apparatus should prevent the user from either missing the skin tag or cutting too deeply into the skin. Moreover, such an apparatus may also be configured to facilitate containment and/or abatement of any bleeding at the wound site.

### II. SUMMARY OF THE INVENTION

Following, a broad object of a particular embodiment of the invention can be to provide a skin growth excision apparatus, and methods of making and using such a skin growth excision apparatus, whereby the skin growth excision apparatus can include a base having opposing base upper and lower surfaces, an aperture element communicating between the base upper and lower surfaces, and a blade overlaying the base upper surface. The blade can be slidably engaged with the base to facilitate travel of the blade along a travel path over the aperture element, whereby the blade can excise a skin growth extending therethrough.

Naturally, further objects of the invention are disclosed throughout other areas of the specification, drawings, and claims.

### III. A BRIEF DESCRIPTION OF THE DRAWINGS

FIG. 1 shows a method of using a particular embodiment of the inventive skin growth excision apparatus.

FIG. 2A shows a top perspective view of a particular embodiment of the inventive skin growth excision apparatus disposed in an open condition in which the blade is located away from the aperture element and proximate the base first end.

FIG. 2B shows another top perspective view of the apparatus illustrated in FIG. 2A.

FIG. 2C shows a bottom perspective view of the apparatus illustrated in FIG. 2A.

FIG. 2D shows another bottom perspective view of the apparatus illustrated in FIG. 2A.

FIG. 2E shows a front view of the apparatus illustrated in FIG. 2A.

FIG. 2F shows a rear view of the apparatus illustrated in FIG. 2A.

FIG. 2G shows a second end view of the apparatus illustrated in FIG. 2A.

FIG. 2H shows a first end view of the apparatus illustrated in FIG. 2A.

FIG. 2I shows a top view of the apparatus illustrated in FIG. 2A.

FIG. 2J shows a bottom view of the apparatus illustrated in FIG. 2A.

FIG. 3A shows a top perspective view of a particular embodiment of the inventive skin growth excision apparatus disposed in a closed condition in which the blade, having traveled over the aperture element, disposes within the cover and proximate the base second end.

FIG. 3B shows another top perspective view of the apparatus illustrated in FIG. 3A.

FIG. 3C shows a bottom perspective view of the apparatus illustrated in FIG. 3A.

FIG. 3D shows another bottom perspective view of the apparatus illustrated in FIG. 3A.

FIG. 3E shows a front view of the apparatus illustrated in FIG. 3A.

FIG. 3F shows a rear view of the apparatus illustrated in FIG. 3A.

FIG. 3G shows a second end view of the apparatus illustrated in FIG. 3A.

FIG. 3H shows a first end view of the apparatus illustrated in FIG. 3A.

FIG. 3I shows a top view of the apparatus illustrated in FIG. 3A.

FIG. 3J shows a bottom view of the apparatus illustrated in FIG. 3A.

FIG. 4A shows a top perspective view of a particular embodiment of a base of the inventive skin growth excision apparatus.

FIG. 4B shows another top perspective view of the base illustrated in FIG. 4A.

FIG. 4C shows a bottom perspective view of the base illustrated in FIG. 4A.

FIG. 4D shows another bottom perspective view of the base illustrated in FIG. 4A.

FIG. 4E shows a front view of the base illustrated in FIG. 4A.

FIG. 4F shows a rear view of the base illustrated in FIG. 4A.

FIG. 4G shows a second end view of the base illustrated in FIG. 4A.

FIG. 4H shows a first end view of the base illustrated in FIG. 4A.

FIG. 4I shows a top view of the base illustrated in FIG. 4A.

FIG. 4J shows a bottom view of the base illustrated in FIG. 4A.

FIG. 5A shows a top perspective view of a particular embodiment of a projection of the inventive skin growth excision apparatus.

FIG. 5B is an enlarged view of a portion of FIG. 5A.

FIG. 6 is an exploded perspective view of a particular embodiment of a blade, a pad, and a blade actuator of the inventive skin growth excision apparatus.

FIG. 7 is a top view of a particular embodiment of a blade actuator, a blade, a protective layer, and a base of the inventive skin growth excision apparatus.

FIG. 8 is an exploded perspective view of a particular embodiment of a blade actuator, a cover, a protective layer, and a base of the inventive skin growth excision apparatus.

FIG. 9 is an exploded perspective view of a particular embodiment of the inventive skin growth excision apparatus.

### IV. DETAILED DESCRIPTION OF THE PREFERRED EMBODIMENTS

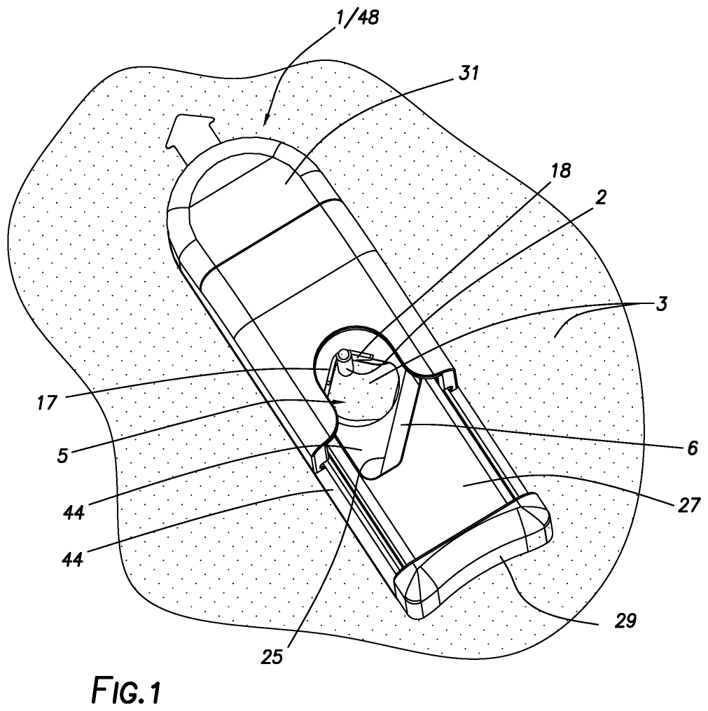

Now referring primarily to FIG. 1, which illustrates a particular embodiment of the inventive skin growth excision apparatus (1) which may be useful for the removal of a skin growth (2) from the skin (3). Generally, the apparatus (1) includes a base (4), an aperture element (5) disposed within the base (4), and a blade (6) overlaying the base (4), whereby the blade (6) can be slidably engaged with the base (4) to facilitate travel of the blade (6) along a travel path (7) in a first direction (8) over the aperture element (5). In use, following receipt of a skin growth (2) within the aperture element (5), the blade (6) can be actuated to slide along the travel path (7) in the first direction (8) over the aperture element (5) and through the skin growth (2) extending therethrough, correspondingly excising or cutting the skin growth (2) proximate its base to effectively detach the skin growth (2) from the skin (3).

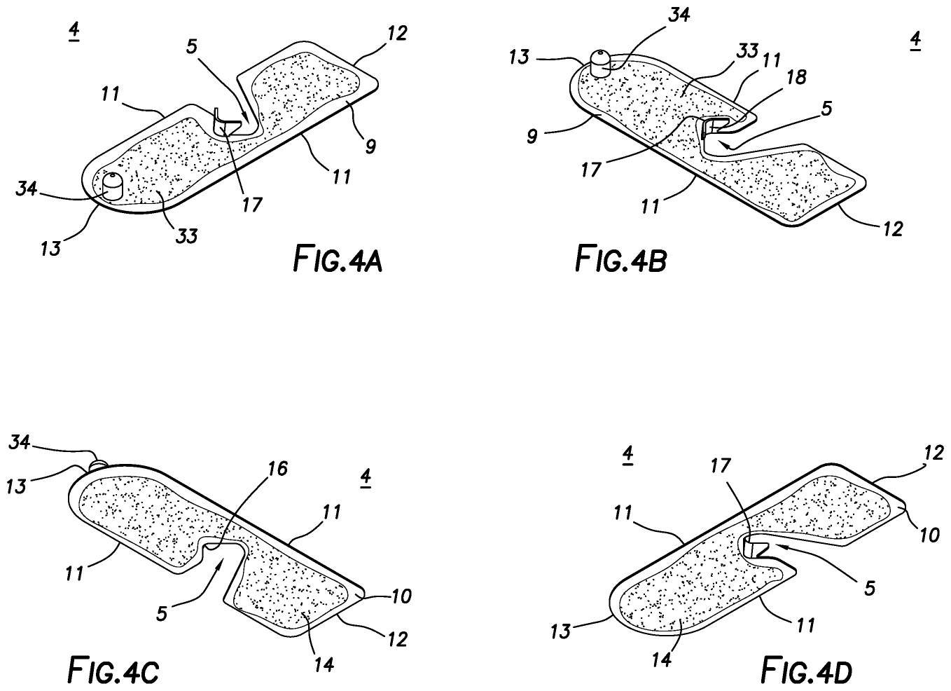

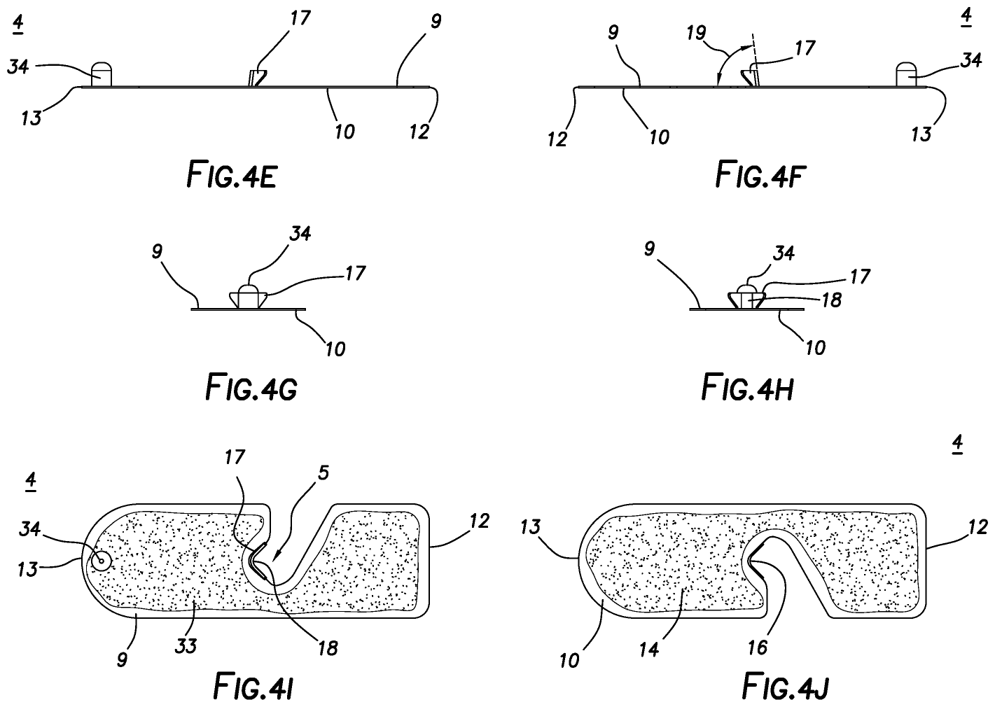

Now referring primarily to FIGS. 4A through 4J, and 9, the inventive skin growth excision apparatus (1) includes a base (4) having opposing base upper and lower surfaces (9)(10) which, as to particular embodiments, can be substantially planar. In addition, the base (4) can be defined by a lateral periphery including opposing base sides (11) and opposing base first and second ends (12)(13). As to particular embodiments, the opposing base sides (11) can be disposed in substantially parallel relation. Concerning the base ends (12)(13), as to particular embodiments, the base first end (12) can be substantially planar and the base second end (13) can be arcuate, such as convexly arcuate; thus, the base ends (12)(13) can be readily distinguishable from one another, which may permit easy identification of directionality to assist with proper placement of the apparatus (1) on the skin (3).

Regarding configuration, as to particular embodiments, the apparatus (1) can have an elongate shape, correspondingly having a longitudinal axis which passes through the base ends (12)(13). As to these embodiments, the apparatus (1) may have a generally rectangular shape, either entirely or in part, whereby the latter may include an arcuate base second end (13).

As to other particular embodiments, the apparatus (1) can have a generally circular shape (not shown), which may be aesthetically pleasing, for example to a consumer, user, or patient.

Now referring primarily to FIGS. 4C, 4D, and 4J, the base (4) can further include a first adhesive (14) (or a first adhesive layer) coupled to the base lower surface (10), whereby the first adhesive (14) can function to couple the base (4) to the body and specifically, to the portion of the skin (3) having the skin growth (2) intended for removal. The first adhesive (14) can be any suitable adhesive, and preferably an adhesive designed for releasable adherence to human skin. Of course, prior to use, the first adhesive (14) can be covered by a removable backing (15) as may be known in the art.

As to particular embodiment, the base (4) can be formed from a flexible material (as opposed to a rigid material), such as a relatively thin layer of plastic, plastic-like material, paper, paper-like material, fabric, or the like, thus allowing the base (4) to conform to the portion of the skin (3) having the skin growth (2) intended for removal.

Now referring primarily to FIGS. 4A through 4D, 4I, and 4J, the apparatus (1) can further include an aperture element (5) disposed within the base (4) to communicate between the base upper and lower surfaces (9)(10).

As to particular embodiments, the aperture element (5) can additionally communicate with a base side (11), thus the aperture element (5) can be considered an open-sided aperture element (5) which allows lateral receipt of the skin growth (2) therein.

As to other particular embodiments, the aperture element (5) can be laterally enclosed (not shown); accordingly, the base (4) can be vertically moved in relation to the skin (3) for receipt of the skin growth (2) within the aperture element (5).

The aperture element (5) can include an aperture element terminal portion (16) positioned on the part of the aperture element (5) nearest the base second end (13), whereby the aperture element terminal portion (16) can be the targeted location for disposition of the skin growth (2) when extending through the aperture element (5) such that the base of the skin growth (2) abuttingly engages with the wall of the base (4) defining the aperture element terminal portion (16).

As to particular embodiments, the aperture element terminal portion (16) can have an arcuate configuration, such as a concavely arcuate configuration, tapering toward the base second end (13); said another way, the aperture element terminal portion (16) may be considered to have a generally U-shape or a generally V-shape. Such an arcuate configuration, as opposed to a linear or straight configuration, may facilitate engagement of the skin growth (2) with the wall of the base (4) defining the aperture element terminal portion (16) and/or preclude movement, such as rolling movement, of the skin growth (2) during excision.

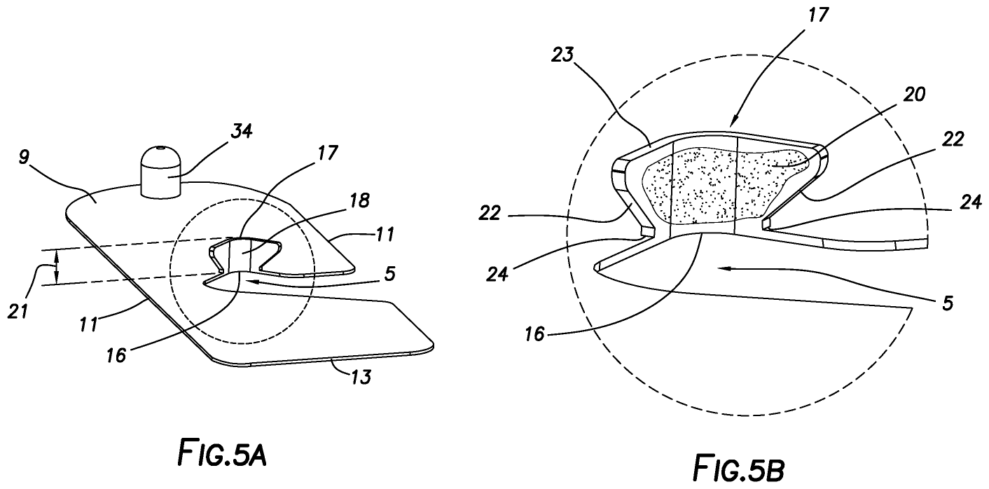

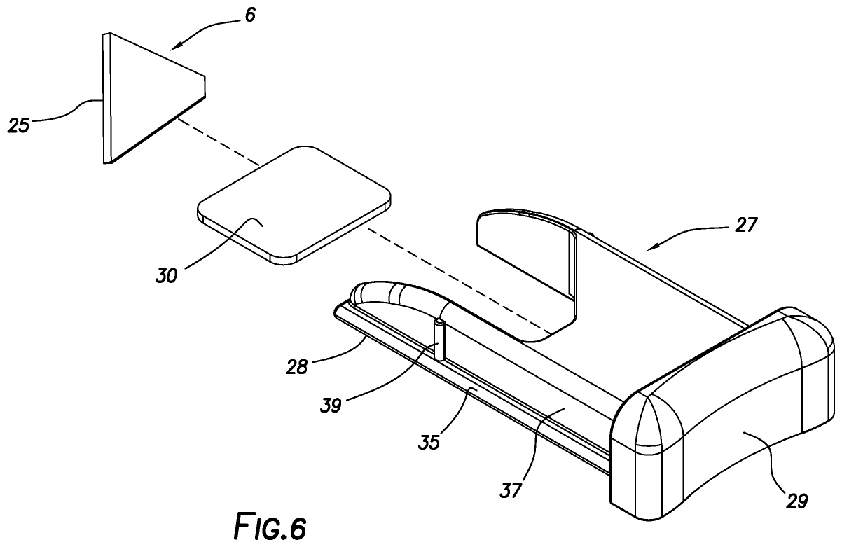

Now referring primarily to FIGS. 5A and 5B, the base (4) can further include a projection (17) upwardly extending from the base upper surface (9) proximate the aperture element (5) and specifically, proximate the aperture element terminal portion (16). The projection (17) can include a projection face (18) disposed toward the aperture element (5), whereby the projection face (18) can dispose in angled relation to the aperture element (5) and/or the base upper surface (9). As to particular embodiments, the projection face (18) can dispose in substantially perpendicular relation to the aperture element (5) and/or the base upper surface (9). As to particular embodiments, the projection face (18) can dispose at an angle (19) of about 90° degrees in relation to the aperture element (5) and/or the base upper surface (9) (as shown in FIG. 4F). As to particular embodiments, the projection face (18) can dispose at an angle (19) of not lesser than about 45° and not greater than about 90° in relation to the aperture element (5) and/or the base upper surface (9). As to particular embodiments, the projection face (18) can dispose at an angle (19) of not lesser than about 80° and not greater than about 90° in relation to the aperture element (5) and/or the base upper surface (9). As but one illustrative example, the projection face (18) can dispose at an angle (19) of about 85° in relation to the aperture element (5) and/or the base upper surface (9).

As the aperture element terminal portion (16) below or beneath the projection face (18) can be the targeted location for the base of a skin growth (2) extending through the aperture element (5), the projection face (18) can be configured to abuttingly engage with a portion of the skin growth (2) above its base to support the skin growth (2) in such a location for excision. Additionally, as to particular embodiments, a second adhesive (20) can be coupled to the projection face (18) (as shown in FIG. 5B); following, the skin growth (2) can adhere to the second adhesive (20), whereby the second adhesive (20) can function to secure the skin growth (2) to the projection face (18) and accordingly, effectively restrict movement of, restrain, or immobilize the skin growth (2) during excision.

As to particular embodiments, the projection face (18) can have an arcuate configuration (such as a concavely arcuate configuration tapering toward the base second end (13)), which may follow the arcuate configuration of the aperture element terminal portion (16). Correspondingly, the projection face (18) may be considered to have a generally U-shape or a generally V-shape. Such an arcuate configuration, as opposed to a linear or straight configuration, may facilitate engagement of a skin growth (2) with the projection face (18) and/or preclude movement, such as rolling movement, of the skin growth (2) during excision. Further, such an arcuate configuration may allow conformation of the skin growth (2) to the protrusion face (18), as many skin growths (2) may typically have a generally cylindrical configuration.

Now referring primarily to FIG. 5A, the projection face (18) can have a projection face height (21) which can be similar to or greater than the height of the majority of skin growths (2); thus, the protrusion face (18) can be sufficient for abuttingly engaging and/or supporting and/or securing the majority of skin growths (2). As but one illustrative example, the projection face height (21) can be about 0.1 inches.

Now referring primarily to FIG. 5B, the projection (17) can terminate in opposing projection lateral edges (22). As to particular embodiments, the projection lateral edges (22) can taper toward the base (4). Regarding this configuration, the projection upper edge (23) can have a greater length than the opposing projection lower edge (24), whereby the projection (17) can be coupled or attached to the base (4) via the projection lower edge (24).

As to particular embodiments, the projection (17) can be removably coupled to the base (4). For example, the projection (17) can be excisable from the base (4); following, the projection (17) can be excised from the base (4) during excision of a skin growth (2) from the skin (3) by the blade (6). As to these particular embodiments, the lesser length of the projection lower edge (24) may be advantageous for such an excision, as the lesser length provides a lesser amount of material for the blade (6) to cut through to excise the projection (17) from the base (4). It is noted that the removable characteristic of the instant projection (17) may be contrasted with a projection intended to be fixedly or permanently coupled to a base, whereby such a projection may function as a stop element to arrest travel of a blade.

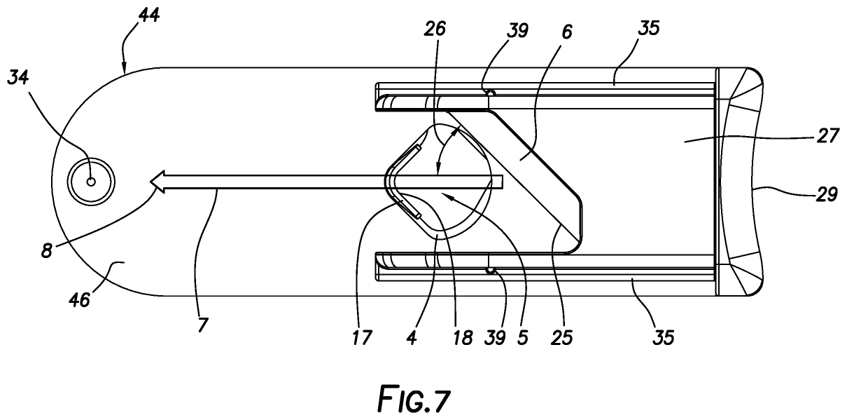

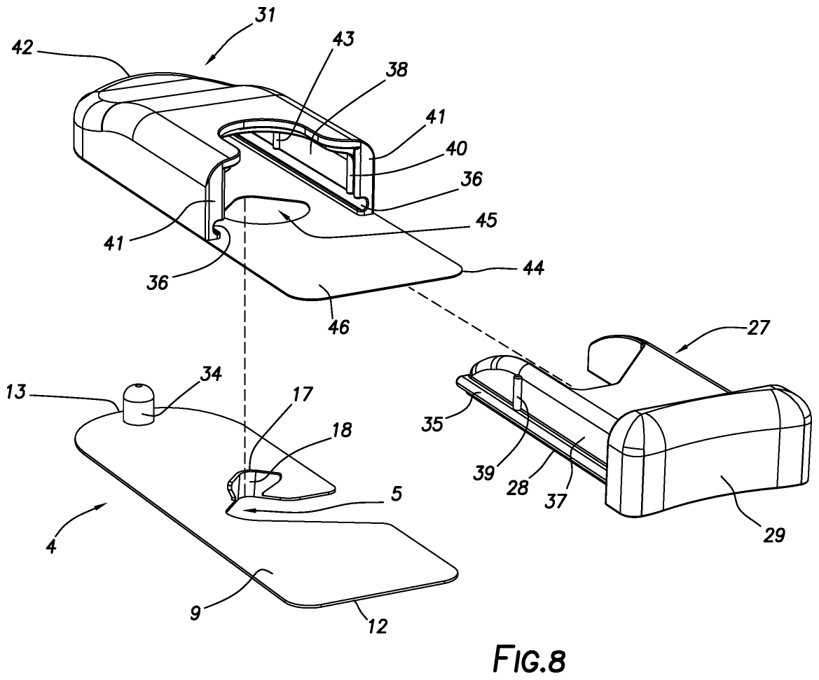

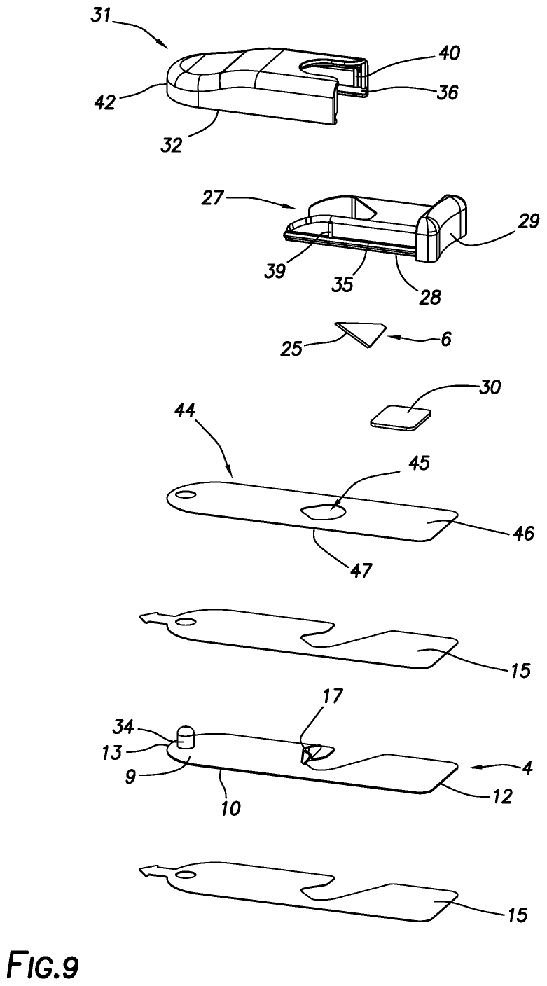

Now referring primarily to FIGS. 6, 7, and 9, the apparatus (1) further includes a blade (6) overlaying the base (4) and/or the base upper surface (9), whereby the blade (6) can be movably engaged with the base (4) such that the blade (6) can travel along a travel path (7) in a first direction (8) over the aperture element (5) (as shown in FIG. 7). The instant blade (6) includes a sharp blade edge (25) which facilitates cutting or slicing.

To reiterate the above, in use, following receipt of a skin growth (2) within the aperture element (5), the blade (6) can be actuated to move (for example, via sliding) along the travel path (7) in the first direction (8) over the aperture element (5) and through the skin growth (2) extending therethrough, correspondingly excising or cutting the skin growth (2) proximate its base to effectively detach the skin growth (2) from the skin (3).

Now referring primarily to FIG. 7, as to particular embodiments, in relation to the base (4), the blade (6) can be positioned such that the blade edge (25) disposes in non-perpendicular relation to the travel path (7). Correspondingly, the blade edge (25) can dispose in non-perpendicular relation to a skin growth (2) received within the aperture element terminal portion (16) and supported by/secured to the projection face (18), which may enhance excision of the skin growth (2) by the blade (6).

As to particular embodiments, the blade edge (25) can dispose at an angle (26) of not lesser than about 10° and not greater than about 80° in relation to the travel path (7).

As to particular embodiments, the blade edge (25) can dispose at an angle (26) of not lesser than about 20° and not greater than about 70° in relation to the travel path (7).

As to particular embodiments, the blade edge (25) can dispose at an angle (26) of not lesser than about 30° and not greater than about 60° in relation to the travel path (7).

As to particular embodiments, the blade edge (25) can dispose at an angle (26) of not lesser than about 40° and not greater than about 50° in relation to the travel path (7).

As but one illustrative example, the blade edge (25) can dispose at an angle (26) of about 45° in relation to the travel path (7).

Now referring primarily to FIGS. 6 through 9, the apparatus (1) can further include a blade actuator (27) fixedly coupled to the blade (6) and accordingly, overlaying the base (4) and/or the base upper surface (9), whereby the blade actuator (27) can facilitate travel of the blade (6) along the travel path (7) in the first direction (8) over the aperture element (5). Regarding relative positioning, the blade (6) can be located proximate a blade actuator lower surface (28).

The blade actuator (27) can include an engagement surface (29) which upwardly extends from the blade actuator lower surface (28); upon the application of pressure to the engagement surface (29) in the first direction (8), for example by one or more fingers of a user, the blade actuator (27) can be moved to correspondingly move or slide the blade (6) along the travel path (7) in the first direction (8) over the aperture element (5) to excise a skin growth (2) extending therethrough. As to particular embodiments, the engagement surface (29) can dispose in substantially perpendicular relation to the blade (6) and/or the blade actuator lower surface (28). Further, as to particular embodiments, the engagement surface (29) can be arcuate, such as concavely arcuate, which may facilitate engagement with the user's finger(s).

Again referring primarily to FIGS. 6 and 9, the apparatus (1) can further include an absorbent pad (30) coupled to the blade (6) and/or the blade actuator (27) and specifically, to the blade actuator lower surface (28) opposite the blade edge (25), whereby the pad (30) can facilitate absorption of fluid(s) following excision of a skin growth (2). As to particular embodiments, the pad (30) can include a coagulant, which may facilitate coagulation of fluid, such as blood, associated with the excision of the skin growth (2).

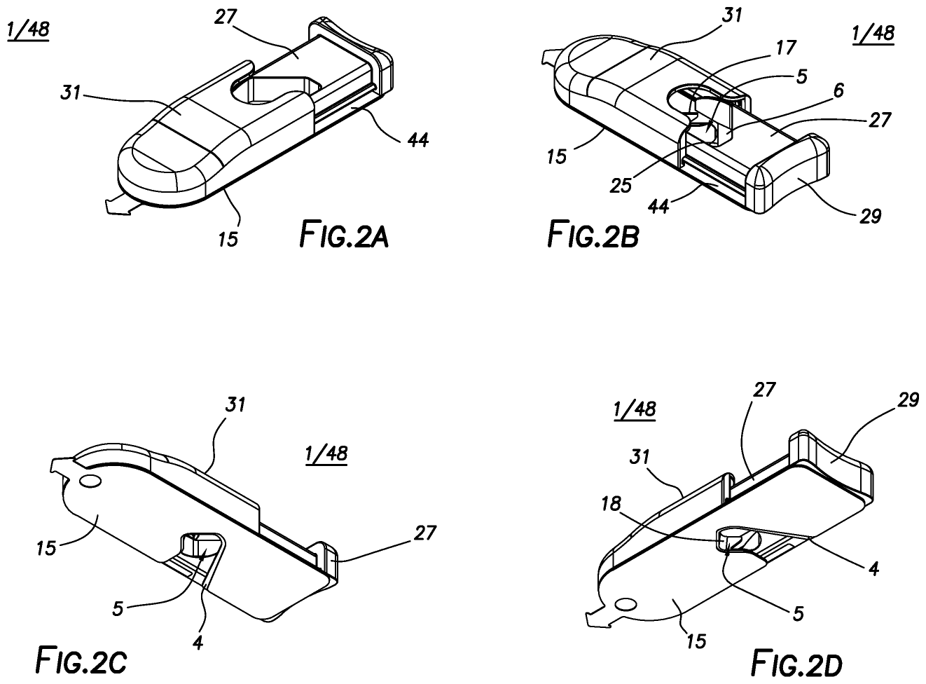

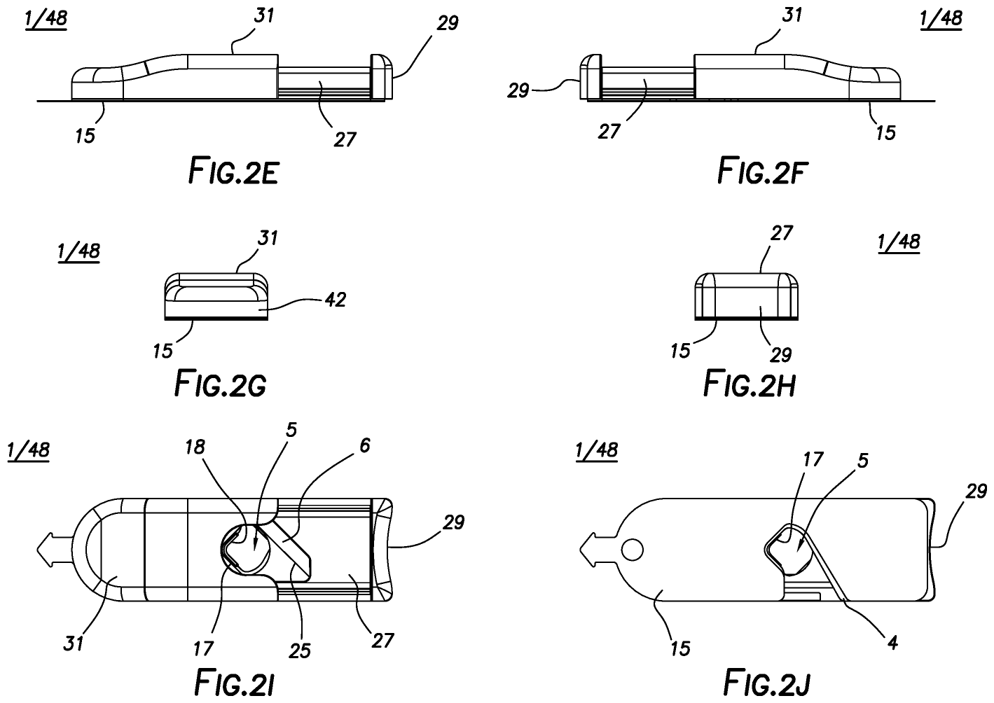

Now referring primarily to FIGS. 2A through 3J, 8, and 9, the apparatus (1) can further include a cover (31) couplable to the base (4), whereby the cover (31) can be provided as a discrete component prior to use of the apparatus (1), meaning a component which is separate from the base (4). Following, in use, after the base (4) has been properly positioned on and secured to the skin (3) such that the skin growth (2) extends through the aperture element (5), the cover (31) can be coupled to the base (4), for example fixedly coupled such that the cover (31) and the base (4) cannot move in relation to one another. In greater detail, a cover lower surface (32) can be coupled to the base upper surface (9); hence, the cover (31) can dispose above the base (4).

As to particular embodiments, a third adhesive (33) can be disposed between the cover lower surface (32) and the base upper surface (9), whereby the third adhesive (33) can function to fixedly couple the cover (31) to the base (4). As to particular embodiments, the third adhesive (33) can be coupled to the base upper surface (9) (as shown in FIGS. 4A, 4B, and 4I). The third adhesive (33) can be any suitable adhesive capable of fixedly adhering the cover (31) to the base (4). Of course, prior to use, the third adhesive (33) can be covered by a removable backing (15) as may be known in the art.

As to particular embodiments, for proper alignment of the cover (31) and the base (4) upon coupling, the cover (31) and the base (4) can include cooperating guide elements intended to guide travel of the cover (31) relative to the base (4). As but one illustrative example, the base (4) can include a post (34) upwardly extending from the base upper surface (9), and the cover (31) can include a corresponding socket disposed therein, whereby the post (34) can be received with the socket to guide proper alignment of the cover (31) with the base (4).

Now referring primarily to FIG. 8, the blade (6) and corresponding blade actuator (27) can be slidably engaged with the cover (31); following, upon fixed coupling of the cover (31) to the base (4), the blade (6) and blade actuator (27) can be slidably engaged with the base (4).

As to particular embodiments, the cover (31) and the blade actuator (27) can be slidably engaged via a tongue and groove system, whereby one of the cover (31) or the blade actuator (27) includes a tongue (35) (or outwardly-extending protrusion) and the other includes a corresponding groove (36) (or inwardly-extending channel). As but one illustrative example, the blade actuator (27) can include a tongue (35) outwardly extending from each of its outer sides (37), and the cover (31) can include a corresponding groove (36) inwardly extending into each of its inner sides (38). Upon receipt of the tongues (35) within the grooves (36), the blade actuator (27) can be slidably engaged with the cover (31).

Again referring primarily to FIG. 8, as to particular embodiments, the cover (31) and the blade actuator (27) can further include cooperating stop elements intended to limit travel of the blade actuator (27) relative to the cover (31) and correspondingly, the base (4).

A wide variety of stop element configurations may be useful with the instant apparatus (1); as but one illustrative example, as to particular embodiments, the blade actuator (27) can include a blade actuator protuberance (39) outwardly extending from its outer side(s) (37), and the cover (31) can include a cooperating cover first protuberance (40) outwardly extending from its inner side(s) (38), whereby the cover first protuberance (40) can be disposed proximate a cover first end (41) into which the blade actuator (27) is received for slidable engagement. For assembly, the blade actuator (27) can be urged toward a cover second end (42) and the blade actuator protuberance (39) can be forced to pass over the cover first protuberance (40); subsequently, the blade actuator protuberance (39) can dispose between the cover first protuberance (40) and the cover second end (42). In use, the cover first protuberance (40) can function to limit travel of the blade actuator (27) in a direction opposite of the first direction (8), thus precluding the blade actuator (27) and correspondingly, the blade (6), from disengaging from the cover (31).

As to particular embodiments, the cover (31) can further include a cover second protuberance (43) which can also cooperate with the blade actuator protuberance (39), whereby the cover second protuberance (43) can outwardly extend from its inner side(s) (38) proximate the cover second end (42). During use, the blade actuator (27) can be urged toward the cover second end (42) for excision of the skin growth (2) extending through the aperture element (5). After the blade (6) cuts through the skin growth (2) and the projection (17), the blade actuator protuberance (39) can be forced to pass over the cover second protuberance (43). Consequently, the blade actuator protuberance (39) can dispose between the cover second protuberance (43) and the cover second end (42). The cover second protuberance (43) can function to limit travel of the blade actuator (27) in a direction opposite of the first direction (8), thus precluding the blade actuator (27) and correspondingly, the blade (6), from disengaging from the cover (31) to effectively lock the blade actuator (27) and the cover (31) together after excision.

Now referring primarily to FIGS. 7 through 9, the apparatus (1) can further include a protective layer (44) disposed between the blade (6) and the base (4), the protective layer (44) including an opening (45) extending therethrough between protective layer upper and lower surfaces (46)(47).

As to particular embodiments, the protective layer (44) can be coupled to the cover (31) and specifically, the protective layer upper surface (46) can be coupled to the cover lower surface (32), for example via an adhesive. Accordingly, upon slidable engagement of the blade actuator (27) with the cover (31), the protective layer (44) can dispose below or beneath the blade (6). Upon fixed coupling of the cover (31) to the base (4), for example via adherence of the third adhesive (33) coupled to the cover lower surface (32) to the protective layer upper surface (46), the protective layer (44) can dispose between the blade (6) and the base (4).

The opening (45) communicating between the protective layer upper and lower surfaces (46)(47) can be configured to vertically align with (i) a portion of the aperture element (5) and in particular, with the aperture element terminal portion (16) and (ii) the projection (17) when the protective layer (44) disposes between the blade (6) and the base (4), the projection (17) correspondingly extending through the opening (45). Following, in use, a skin growth (2) engaged with the projection (17) correspondingly extends through the opening (45), thus allowing excision of the skin growth (2) and projection (17) upon actuation of the blade (6) while protecting the surrounding skin (3) from the blade (6) to minimize the risk of unnecessary injury to said skin (3).

### Production

Regarding production, a method of making the inventive skin growth excision apparatus (1) can include providing a base (4) comprising opposing base upper and lower surfaces (9)(10) and an aperture element (5) disposed therein to communicate between the base upper and lower surfaces (9)(10), and slidably engaging a blade (6) with the base (4), the blade (6) overlaying the base upper surface (10). As detailed above, the blade (6) can be configured to travel along a travel path (7) in a first direction (8) over the aperture element (5) to excise a skin growth (2) extending therethrough.

The method of making particular embodiments of the skin growth excision apparatus (1) can further include providing and adding additional components of the apparatus (1) as described above and in the claims.

Concerning materials, as detailed above, the base (4) can be formed from a flexible material, such as a relatively thin layer of plastic, plastic-like material, paper, paper-like material, fabric, or the like. Additionally, the protective layer (44) can be formed from a similar material, which may also be flexible. As to particular embodiments, the protective layer (44) may be transparent or translucent, which may allow a user to visualize the base (4) to facilitate coupling of the cover (31) with the base (4).

As to particular embodiments, the projection (17) can be integrally formed with the base (4) such that the projection (17) and the base (4) can be formed as a one-piece construct, a monolithic construct, or a unified whole.

The blade (6) can be formed from a material which provides a sharp blade edge (25), such as metal.

Each of the blade actuator (27) and the cover (31) can be formed from a rigid material (as opposed to a flexible material), which may be the same material, whereby the rigidity of the material may enhance excision of the skin growth (2) by the blade (6). As but one illustrative example, the blade actuator (27) and the cover (31) can be formed from plastic or plastic-like material.

As to particular embodiments, the cover (31) can be formed from an opaque material, which may be advantageous for concealing the excised skin growth (2) and/or the wound site post excision.

Concerning making, one or more of the components of the skin growth excision apparatus (1) can be made by molding, injection molding, additive printing (such as three-dimensional (3D) printing), or the like.

As to particular embodiments, the skin growth excision apparatus (1) can be sterilized and subsequently provided as a sterile package.

As to particular embodiments, the skin growth excision apparatus (1) can be provided as a kit, which may additionally include a local anesthetic for topical application prior to excision.

### Use

Regarding employment, a method of using the inventive skin growth excision apparatus (1) to excise a skin growth (2) from the skin (3) can include acquiring a base (4) comprising opposing base upper and lower surfaces (9)(10) and an aperture element (5) disposed therein to communicate between the base upper and lower surfaces (9)(10), coupling the base (4) to the skin (3) such that the skin growth (2) extends through the aperture element (5), and urging a blade (6) along a travel path (7) over the aperture element (5) to excise the skin growth (2) from the skin (3).

Now referring primarily to FIG. 2A through FIG. 2J, prior to use, the apparatus (1) can dispose in an open condition (48) in which the blade (6) is located away from the aperture element (5) and proximate the base first end (12).

For excising, the blade (6) can be urged to travel along the travel path (7) in a first direction (8) toward the base second end (13), thereby cutting or slicing a skin growth (2) extending through the aperture element terminal portion (16).

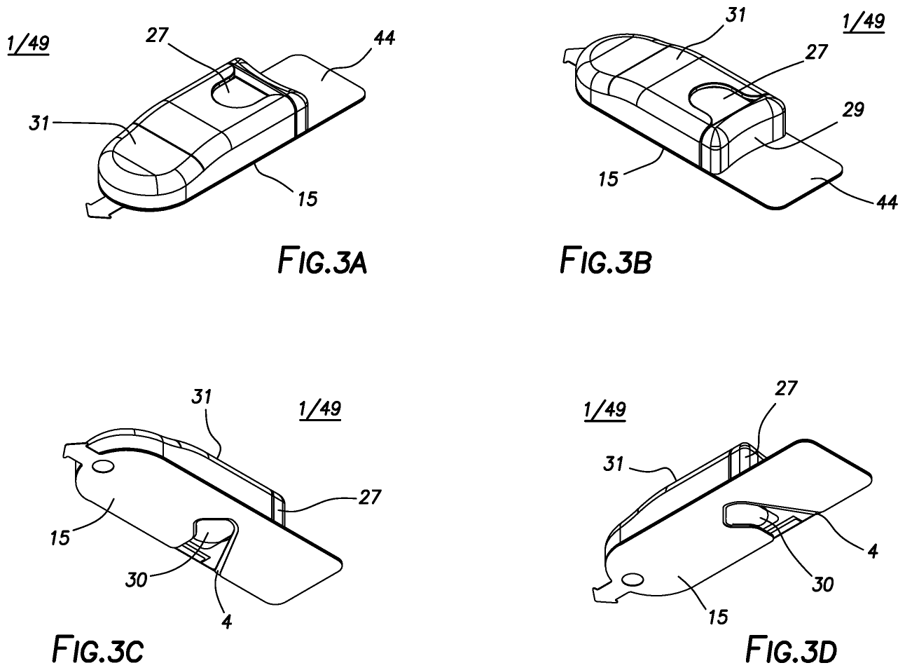

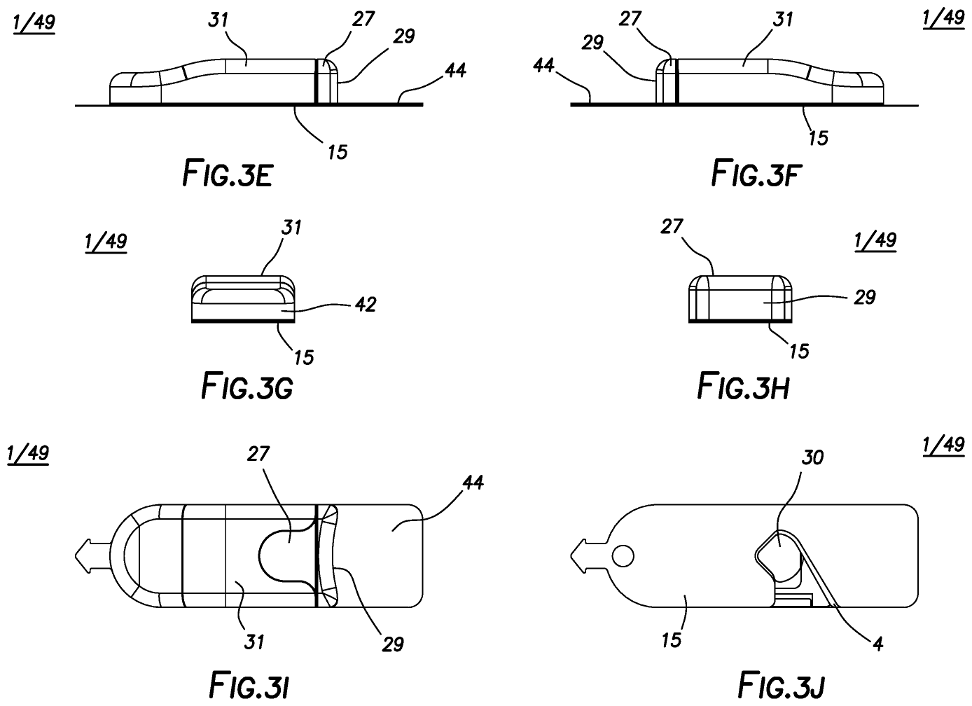

Now referring primarily to FIG. 3A through FIG. 3J, following excision, the apparatus (1) can dispose in a closed condition (49) in which the blade (6), having traveled over the aperture element (5), disposes within the cover (31) and proximate the base second end (13). As to particular embodiments, when in the closed condition (49), the blade actuator (27) and the cover (31) can be locked together, thereby locking the blade (6) within the cover (31).

The method of using particular embodiments of the skin growth excision apparatus (1) can further include employing additional components of the apparatus (1) as described above and in the claims.

As to particular embodiments, the skin growth excision apparatus (1) can be intended as a single-use product, which can be discarded after one use.

As to particular embodiments, the skin growth excision apparatus (1) can be configured for containment of the skin growth (2) following excision. Subsequently, the skin growth excision apparatus (1) containing the excised skin growth (2) can be submitted, for example to a laboratory, for consequent analysis of the skin growth (2).

As can be easily understood from the foregoing, the basic concepts of the present invention may be embodied in a variety of ways. The invention involves numerous and varied embodiments of a skin growth excision apparatus and methods for making and using such a skin growth excision apparatus.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “projection” should be understood to encompass disclosure of the act of “projecting”—whether explicitly discussed or not—and, conversely, were there effectively disclosure of the act of “projecting”, such a disclosure should be understood to encompass disclosure of a “projection” and even a “means for projecting”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Further, for the purposes of the present invention, the term “coupled” or derivatives thereof can mean indirectly coupled, coupled, directly coupled, connected, directly connected, or integrated with, depending upon the embodiment.

Additionally, for the purposes of the present invention, the term “integrated” when referring to two or more components means that the components (i) can be united to provide a one-piece construct, a monolithic construct, or a unified whole, or (ii) can be formed as a one-piece construct, a monolithic construct, or a unified whole. Said another way, the components can be integrally formed, meaning connected together so as to make up a single complete piece or unit, or so as to work together as a single complete piece or unit, and so as to be incapable of being easily dismantled without destroying the integrity of the piece or unit.

Thus, the applicant(s) should be understood to claim at least: i) each of the skin growth excision apparati herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A skin growth excision apparatus, comprising:
a base having opposing base upper and lower surfaces;
an aperture element communicating between said base upper and lower surfaces; and
a blade overlaying said base upper surface, said blade slidably engaged with said base to facilitate travel of said blade along a travel path over said aperture element.

2. The apparatus of claim 1, further comprising a first adhesive coupled to said base lower surface.

3-4. (canceled)

5. The apparatus of claim 1, said aperture element comprising an open-sided aperture element.

6. (canceled)

7. The apparatus of claim 1, said aperture element comprising an aperture element terminal portion.

8. The apparatus of claim 7, said aperture element terminal portion comprising an arcuate configuration tapering toward a base second end of said base.

9. The apparatus of claim 7, further comprising a projection upwardly extending from said base upper surface proximate said aperture element terminal portion.

10. The apparatus of claim 9, said projection comprising a projection face disposed toward said aperture element.

11-14. (canceled)

15. The apparatus of claim 10, further comprising a second adhesive coupled to said projection face.

16. The apparatus of claim 10, said projection face comprising an arcuate configuration tapering toward a base second end of said base.

17-20. (canceled)

21. The apparatus of claim 10, said projection excisable from said base by said blade.

22. The apparatus of claim 1, said blade including a blade edge positioned in non-perpendicular relation to said travel path.

23. The apparatus of claim 22, said blade edge disposed at an angle of not lesser than about 10° and not greater than about 80° relative to said travel path.

24-26. (canceled)

27. The apparatus of claim 10, further comprising a blade actuator fixedly coupled to said blade, said blade actuator configured to facilitate travel of said blade along said travel path in a first direction.

28. The apparatus of claim 27, further comprising an absorbent pad coupled to said blade actuator behind said blade.

29. (canceled)

30. The apparatus of claim 27, further comprising a cover couplable to said base.

31. The apparatus of claim 30, said cover provided as a discrete component from said base prior to use of said apparatus.

32. The apparatus of claim 30, further comprising a third adhesive disposed between said cover and said base upper surface.

33. (canceled)

34. The apparatus of claim 30, said cover slidably engaged with said blade actuator.

35-36. (canceled)

37. The apparatus of claim 34, further comprising a protective layer disposed between said blade and said base, said protective layer including an opening extending therethrough.

38. (canceled)

39. The apparatus of claim 1, said blade sterile.

40-94. (canceled)
